(19) United States
(12) Patent Application Publication
(10) Pub. No.: US 2003/0129257 A1
(43) Pub. Date: Jul. 10, 2003

(54) Polymer-based material comprising silica particles

(75) Inventors: Berthold Nies, Fraenkisch-Crumbach (DE); Matthias Koch, Wiesbaden (DE); Holger Winkler, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 10/314,448

(22) Filed: Dec. 9, 2002

(30) Foreign Application Priority Data
Dec. 7, 2001 (DE) 101 60 329.0

(51) Int. Cl. A61K 33/00

(52) U.S. Cl. 424/724

(57) ABSTRACT

The invention describes a process for the preparation of a polymer-based material consisting of a polyolefin matrix and silica particles as filling material, in which the silica particles are incorporated during the polymerization process. In addition, the uses of a polymer-based material for the production of joint implants in the medicotechnical area and also the use for the production of sliding bearings, gearwheels and other applications in the industrial sector are described. The invention furthermore describes an artificial joint of a prosthesis in which the joint partners consist of metallic, polymeric and/or ceramic materials, with at least one of the joint partners consisting of a polymeric material made from polyolefins with addition of from 1 to 50% by weight of SiO₂.

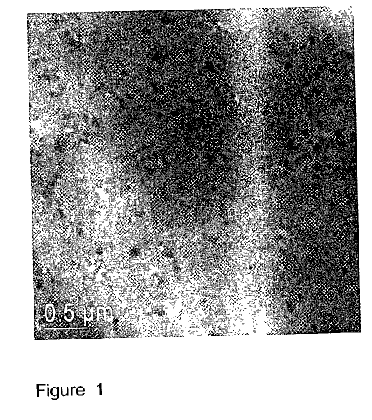

Artificial joints and joint implants use various materials and material combinations which differ, in particular, in price and durability. Combinations of a suitable metal alloy with ultrahigh-molecular-weight polyethylene (UHMWPE) are amongst the inexpensive approaches which make up the bulk of the implants. These “metal-on-polymer” joint implants are described, for example, in WO 01/17464 (Depuy Int. Ltd.).

A disadvantage of the metal/UHMWPE combination is the static friction which exists between the metal and the plastic, making the plastic part susceptible to wear, and the sensitivity of the UHMWPE to fitting errors and damage during fitting, for example by contamination with bone particles or bone cement.

The object of the present invention is therefore to provide a polymer-based material and a process for the preparation thereof which at least partially overcomes the disadvantages of the prior art through the material enabling reduced friction between two joint parts in joint implants or artificial joints.

This object is achieved by a polymer-based material comprising a polyethylene matrix wherein monospherical SiO₂ particles functionalized with non-acidic nucleophiles are distributed in a nanodisperse and homogenous manner.

Also part of the invention is a process for making a polymer-based material comprising a polyolefin matrix and silica particles as a filling material, said process comprising incorporating said silica particles as early as during the polymerization process, wherein said silica particles are functionalized with non-acidic nucleophiles. Moroever, the invention includes the uses of a polymer-based material for the production of joint implants (for example hip and knee joints) in the medicotechnical area and also the use for the production of sliding bearings, gearwheels and other applications in the industrial sector.

The present invention furthermore relates to an artificial joint of a prosthesis in which the joint partners consist of metallic, polymeric and/or ceramic materials, with at least one of the joint partners consisting of a polymeric material made from polyolefins with addition of from 1 to 50% by weight of SiO₂. A range from 5 to 30% by weight of SiO₂ is preferred.

Suitable polyolefin matrices are all types of polyolefins. However, preference is given to polyethylenes (PE).

The PE moldings used are preferably UHMWPE moldings, which generally have an extremely high molecular weight of between 3.9 and 10.5 million g/mol. Some technically important properties of the polyethylenes, such as, for example, wear resistance, notched impact strength, dimensional stability on heating and anti-friction properties, improve with increasing molecular weight. At the same time, the processing properties of the high-molecular-weight PEs are impaired. It is therefore also an object of the invention to be able to use a PE with good processing properties in the composites with similarly good or better properties than with the PEs conventional hitherto.

In prostheses with artificial joints, the requisite compatibility with human or animal tissue, so-called biocompatibility, and the high frictional stress of the joint partners mean that only few materials can be employed. Of the metallic materials, cobalt/chromium/molybdenum (CoCrMo) in particular has proven successful. Applications of titanium-based alloys or surface-modified titanium alloys (for example titanium nitride) are still at the experimental stage. Of the plastics, use is made, in particular, of polyethylene (UHMWPE) in the sockets of hip-joint endoprostheses and as sliding partners of the shinbone component, and aluminium oxide and zirconium oxide are the ceramic materials which can be used.

Wear within the artificial joint is minimized by the incorporation of hard, non-porous SiO₂ beads or silica particles into the PE molding.

In order to achieve a nanodisperse and extremely homogeneous distribution of the silica particles in the PE, it is advantageous to incorporate them as early as during the polymerization process. UHMWPE is usually not extruded, but instead processed by hot pressing. Subsequent admixing of fillers is not readily possible in this process.

Various routes (or combinations thereof) can be followed for the incorporation of silica particles into PE during the polymerization process with suitable functionalizations. The silica particles can be loaded, for example, with metallocenes and then employed for the polymerization. It is likewise possible to replace the metallocene loading with a polymerization-active Ziegler-Natta species (for example TiCl₄) or Phillips species (for example, chromium oxide immobilized on silica). In this case, the PE forms around the particles, which effects better distribution than subsequent mixing during the extrusion process. It is preferable to select the loading ratio in such a way that the silica component is present in the polyolefin in an amount of between 1 and 50% by weight of SiO₂, based on the total weight of the mixture. It should preferably be between 5 and 30% by weight. However, an excessively high SiO₂ content (>55% by weight) has the disadvantage that the material becomes brittle and can fracture.

Functionalization of the silica particles may be with nucleophilic, non-acidic compounds. Such materials include all polyethers with n=1 to 10 and more, also branched ones such as (CH₂)ₙ(CH((CH₂)ₘCH₃))—O with n<1, and m≦0. Preferred are linear ones with n=2 to 4. Representative polyethers include polyethyleneglycol, polypropenyleneoxide, and Poly-THF. For example, functionalization may be with polyethers, which enables direct support of methylaluminoxane (MAO)-activatable compounds, principally those of metallocenes (as described in German application DE 10160328.2 and in WO 00/78823). The functionalized particles form a gel together with MAO by means of coordinative interactions. The metallocene is thus bound to the support via the MAO. A homogeneous distribution of support and polymerization-active species is achieved in the process. MAO is a cocatalyst that is used to activate metallocenes for olefin polymerization. It generates the polymerization active cationic species from a metallocene. Herein, in this embodiment, the term “catalyst” refers to the activate metallocene-MAO-complex. After drying, the catalyst can be employed directly for the polymerization. In Ziegler-Nafta catalysts (e.g., TiCl₄ immobilized on MgCl₂), the polymerization-active compound is applied directly (for example as TiCl₄ vapor) to the silica particles by generally known methods.

The polymerization is carried out continuously or discontinuously in a known manner in solution, suspension or in the gas phase, with gas-phase and suspension polymerization expressly being preferred. Typical temperatures in the polymerization are in the range from 0° C. to 200° C., preferably in the range from 20° C. to 140° C. Further details on the preparation process are given in German application DE 101 60328.2.

Metallocene loading and catalyst activation are selected in such a way that a catalyst productivity of between 2 and 10 (g of PE)/(g of cat.) is achieved. This corresponds to a silica content of from 10 to 50% by weight.

Catalysts which are usually employed for polyolefin production and which are designed for high productivities can likewise be used through “dilution” with correspondingly treated silica particles. Preference is given here to the use of olefin-functionalized particles whose surface no longer carries any reactive groups. The deactivation is carried out firstly by the olefin functionalization and in addition by treatment with alkylaluminium compounds, for example diisobutylaluminium hydride.

Alternatively, it is possible to carry out anchoring of the silica particles in the PE by alkylic or olefinic functions on the SiO₂, which are incorporated into the PE as copolymers in order to achieve particularly good anchoring. The olefinic functions used are molecules such as

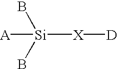

where A is an alkoxy group, preferably ethoxy or methoxy, B is an alkoxy or alkyl group of the same type, preferably methyl, and D is a hydrocarbon having at least one terminal alkyl or alkenyl function, preferably a linear alkenyl chain having a terminal double bond.

X is either a direct bond or a heteroatom-containing link, such as (CH₂)ₙ—O—, where n is between 1 and 5.

For the alkyl functions, use is made of molecules such as

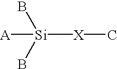

where A is an alkoxy group, preferably ethoxy or methoxy, B is an alkoxy or alkyl group of the same type, preferably methyl, and C is an alkyl chain having a length of 1-25, preferably 10-20.

X is either a direct bond or a heteroatom-containing link, such as (CH₂)ₙ—O—, where n is between 1 and 5.

The following examples and the figure are intended to explain the invention in greater detail.

### BRIEF DESCRIPTION OF THE DRAWINGS

The FIGURE shows a TEM photomicrograph (recorded using a Philips CM20 transmission electron microscope, acceleration voltage 200 kV) of a PE comprising 8% by weight of silica, the sample having been prepared by ultramicrotome technology. The material was prepared with the aid of metallocene-loaded silica particles. The catalyst productivity was 11 g of polyethylene/g of catalyst. The distribution of the silica particles can clearly be seen.

The monospheres (=monodisperse SiO₂ particles) functionalized with non-acidic nucleophiles, such as polyether, preferably with PEO, have a diameter of from 10 to 500 nm, preferably about 100 nm. The amount of metallocene immobilised on the monospheres is selected in such a way that sufficient polyolefin, preferably UHMWPE, is formed during the polymerization in order to keep the silica content of the product at between 1 and 50% by weight, preferably between 5 and 30% by weight.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLES

1. Use as Microparticulate Material for Metallocenes

2 g of polyethylene oxide-functionalized monospheres (for preparation, see parallel application “Microparticulate Material” by the applicant with the same application date) having a diameter of 100 nm were stirred for two hours with 4 ml of a solution of methylaluminoxane in toluene (w(MAO)=5%), and 0.2 ml of a solution of dicyclopentadienylzirconium dichloride in the above MAO solution (c(Zr)=0.05 mol/l) was added. After stirring had been continued for a further 30 minutes, the solvent was removed under reduced pressure, leaving a yellow flowing powder which was employed directly for the polymerization (loading: 2.3 μmol of Zr/g of cat., Al/Zr=630).

For the polymerization, 960 mg of the above catalyst were introduced at 70° C. through a lock by means of an excess pressure of argon into a 1 l steel reactor filled with 400 ml of isobutane and pressurised to 36 bar with ethene. After 10 minutes, the polymerization was terminated by releasing the pressure. Yield: 8.6 g of colourless, flowable powder (productivity: 7.6 g of PE/g of cat., silica content: 11%).

The product exhibits increased abrasion resistance in the ASTM G99 abrasion test compared with conventional PE.

2. Use as Reactor Admixture

Preparation of C₁₈-silanised Monospheres

132 g of monosphere dispersion and 150 g of ethanol are introduced into the 500 ml three-necked flask. The mixture is heated to 70° C., and 12.7 g of ethanolic silane solution are added dropwise over a period of 20 minutes by means of the dropping funnel with vigorous stirring. During the addition, it must be ensured that the solution is introduced at the edge of the vessel, but does not come into contact with its wall.

The mixture is then refluxed for 20 hours. The suspension, in which the solid is in conglomerated form, is transferred into a beaker. The solid is washed five times with deionised water by decantation. After addition of toluene, the water is removed azeotropically from the mixture by means of a water separator.

Gravimetric weight determination gives an SiO₂ concentration of the dispersion of 19.4% by weight.

Use as Reactor Admixture

10 g of the octadodecyl-functionalized monospheres prepared above in 400 ml of isobutane are introduced into a 1 l steel reactor. The reactor is heated to 70° C. and pressurised to 36 bar with ethene. 80 mg of silica-supported dicyclopentadienylzirconium dichloride are introduced through a lock by means of an excess pressure of argon. After 60 minutes, the reaction is terminated by releasing the pressure.

Yield: 78 g of colorless granules (0.5-1 mm particle diameter). Catalyst productivity: 850 g of PE/g of catalyst, silica content: 12.8% by weight. The product exhibits increased abrasion resistance in the ASTM G99 abrasion test compared with PE prepared by an identical method without the admixture.

The entire disclosures of all applications, patents and publications, cited herein and of corresponding German application No. DE 101 60 329.0, filed Dec. 7, 2001 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A polymer-based material comprising a polyethylene matrix wherein monospherical SiO₂ particles functionalized with non-acidic nucleophiles are distributed in a nanodisperse and homogeneous mannner.

2. A polymer-based material according to claim 1, wherein the SiO₂ particles have been functionalized with polyether chains.

3. A polymer-based material according to claim 1, wherein the SiO₂ particles have been functionalized with polyethylene oxide chains.

4. A polymer-based material according to claim 1, wherein alkyl- or olefin-functionalized silica particles are employed.

5. A polymer-based material according to claim 1, wherein the SiO₂ particles have a diameter of 100 nm.

6. A polymer-based material according to claim 3, wherein the SiO₂ particles have a diameter of 200 nm.

7. A polymer-based material according to claim 1, having a content of SiO₂ particles of 1 to 50%.

8. A joint implant comprising a polymer-based material according to claim 1.

9. A sliding bearing or gearwheel comprising a polymer-based material according to claim 1.

10. An artificial joint of a prosthesis having joint partners of metallic, polymeric and/or ceramic materials, wherein at least one of the joint partners is a polymeric material according to claim 1.

11. An artificial joint according to claim 10, wherein the polymeric material is a polyolefin having 5 to 30% by weight of SiO₂.

12. An artificial joint according to claim 10, wherein the polymeric material is UHMW polyethylene.

13. An artificial joint according to claim 10, wherein the joint is an artificial hip or knee joint.

14. A process for the preparation of a polymer-based material comprising a polyolefin matrix and silica particles as a filling material, said process comprising incorporating said silica as early as during the polymerization process, wherein said silica particles are functionalized with non-acidic nucleophiles.

15. A process according to claim 14, wherein polyethylene oxide-functionalized silica monospheres having a diameter of 100 nm are used as a metallocene support, and an amount of metallocene immobilized on the monospheres is selected in such a way that sufficient polyolefin is formed during the polymerization in order to keep silica content of the product at 1 to 50% by weight.

16. A process according to claim 14, wherein a polymerisation-active Ziegler-Natta or Phillips species is applied to the silica particles.

17. A process according to claim 14, wherein alkyl-functionalized monospheres are admixed with a catalyst, and employed for polymerization.

18. A process according to claim 14, wherein olefin-functionalized silica monospheres are admixed before polymerisation.
